(19) United States
(12) Patent Application Publication
(10) Pub. No.: US 2016/0016862 A1
(43) Pub. Date: Jan. 21, 2016

(54) Methods and Systems for Forming a Hydrocarbon Product

(71) Applicant: Seerstone LLC, Provo, UT (US)

(72) Inventor: Dallas B. Noyes, Provo, UT (US)

(73) Assignee: Seerstone LLC, Provo, UT (US)

(21) Appl. No.: 14/775,883

(22) PCT Filed: Mar. 12, 2014
(86) PCT No.: PCT/US2014/025091

§ 371 Date: Sep. 14, 2015

Related U.S. Application Data
(60) Provisional application No. 61/788,833, filed on Mar. 15, 2013.

(51) Int. Cl. C07C 2/76; B01J 8/02; B01J 8/08; B01J 8/18; C07C 1/12; B01J 8/24

(52) U.S. Cl. CPC C07C 2/76; C07C 1/12; B01J 8/24; B01J 8/087; B01J 8/1836; B01J 8/0285; B01J 2208/00106; B01J 2208/027

(57) ABSTRACT

A method of forming a hydrocarbon product comprises reacting at least one carbon oxide and at least one lower hydrocarbon in the presence of a plurality of catalyst-containing structures each comprising a nanofiber bound to at least one catalyst nanoparticle to form at least one higher hydrocarbon. Other methods of forming a hydrocarbon are also disclosed, as is a system forming a hydrocarbon product.

### PRIORITY CLAIM

This application claims the benefit of the filing date of U.S. Provisional Patent Application Ser. No. 61/788,833, filed Mar. 15, 2013, for “Methods and Systems for Forming a Hydrocarbon Product,” the disclosure of which is hereby incorporated herein in its entirety by this reference.

### TECHNICAL FIELD

Embodiments of the disclosure relate generally to methods and systems for forming a hydrocarbon product. More specifically, the embodiments of the disclosure relate to methods and systems of forming a higher hydrocarbon from a lower hydrocarbon and a carbon oxide in the presence of a catalyst.

### BACKGROUND

Large reserves of natural gas, which includes methane (CH₄) and other alkanes, have been discovered throughout the world. Significantly, as world reserves of crude-oil-based feedstocks decline, natural gas has been identified as a potential alternative source of transportable fuel. However, at least due to the expenses frequently associated with the handling and transporting large volumes of natural gas (e.g., construction and maintenance of gas pipeline infrastructures, gas compression and storage in vehicles, etc.), utilization of natural gas discovered or produced at remote locations is often economically unfeasible.

One approach to increasing the economic feasibility of using natural gas has been to convert alkanes contained in natural gas (e.g., CH₄) into higher hydrocarbons that can be more easily handled and transported. For example, a combination of synthesis gas and Fischer-Tropsch processing has been used to convert such alkanes into higher hydrocarbons, including n-paraffins, alcohols, and olefins. In such processing, synthesis gas production technologies (e.g., steam methane reforming, partial oxidation, autothermal reforming, gas heated reforming, or combinations thereof) are used to convert alkanes (e.g., CH₄) into carbon monoxide (CO) and hydrogen (H₂), which are then reacted in the Fischer-Tropsch process to form the higher hydrocarbons. For example, using a CH₄ feedstock, the formation of higher hydrocarbons may proceed according to the following equations:

CH₄+H₂O⇄CO+3H₂ (1)

nCO+(2n+1)H₂⇄CₙH₍₂ₙ₊₂₎+nH₂O (2)

Disadvantageously, however, the production of synthesis gas can represent a large fraction of the costs for such conversion processes, requiring substantial equipment, energy, and material expenditures. In addition, the combination of synthesis gas and Fischer-Tropsch processing typically also requires significant expense to separate and dispose of carbon dioxide (CO₂), either already present in the natural gas or formed during synthesis gas production, which tends to lower the conversion efficiency of the Fischer-Tropsch process.

It would be desirable to have new methods and systems for forming higher hydrocarbons from lower hydrocarbons found in natural gas, such as CH₄. It would further be desirable if the new methods and systems were amiable to using CO₂ to form the higher hydrocarbons, facilitated increased conversion efficiency, and were relatively inexpensive and simple in operation.

### DISCLOSURE

Embodiments described herein include methods and systems for forming a hydrocarbon product. For example, in accordance with one embodiment described herein, a method of forming a hydrocarbon product comprises reacting at least one carbon oxide and at least one lower hydrocarbon in the presence of a plurality of catalyst-containing structures each comprising a nanofiber bound to at least one catalyst nanoparticle to form at least one higher hydrocarbon.

In additional embodiments, a method of forming a hydrocarbon product comprises heating at least one carbon oxide and at least one lower hydrocarbon to a temperature within a range of from about 500° C. to about 1200° C. to form at least one heated carbon oxide and at least one heated lower hydrocarbon. The at least one heated carbon oxide and the at least one heated lower hydrocarbon are reacted in the presence of a catalyst to form a reaction product stream comprising at least one higher hydrocarbon. The at least one higher hydrocarbon is separated from other components of the reaction product stream.

In yet additional embodiments, a system for forming a hydrocarbon product comprises at least one heating system, at least one reactor, and at least one separator. The at least one heating system is configured to increase the temperature of at least one carbon oxide and at least one lower hydrocarbon to a temperature within a range of from about 500° C. to about 1200° C. to form at least one heated carbon oxide and at least one heated lower hydrocarbon. The at least one reactor is configured to convert the at least one heated carbon oxide and the at least one heated lower hydrocarbon in the presence of a plurality of catalyst-containing structures each comprising a nanofiber bound to at least one catalyst nanoparticle to form at least one higher hydrocarbon. The at least one separator is positioned and configured to separate the at least one higher hydrocarbon from at least one other material.

### BRIEF DESCRIPTION OF THE DRAWING

FIG. 1 is a simplified schematic view of a system for forming a hydrocarbon product, in accordance with an embodiment of the present disclosure.

### MODE(S) FOR CARRYING OUT THE INVENTION

Methods and systems for forming (e.g., synthesizing) a hydrocarbon product are described. A lower hydrocarbon may be reacted with a carbon oxide under predetermined reaction conditions (e.g., temperatures, pressures, etc.) and in the presence of a catalyst to form a higher hydrocarbon. The reaction may also produce at least one other material, such as water. At least one source gas stream including the lower hydrocarbon and the carbon oxide may be heated to form at least one heated source gas stream. Components of the heated source gas stream may be reacted in the presence of a catalyst to form the higher hydrocarbon. In some embodiments, at least a portion of the catalyst includes catalyst nanoparticles bound to solid structures. The solid structures and the higher hydrocarbon may be formed simultaneously, non-simultaneously, or a combination thereof. The higher hydrocarbon may be separated from one or more additional materials and may be utilized as desired. The higher hydrocarbon may be more valuable, may be more easily handled and transported than the lower hydrocarbon, may have higher energy density than the lower hydrocarbon, and may have enhanced utility as a chemical process feedstock as compared to the lower hydrocarbon. The methods and systems of the disclosure may be more efficient (e.g., reducing equipment and energy requirements, increasing conversion efficiency, etc.) as compared to conventional higher hydrocarbon production technologies. The methods and systems of the disclosure may also be utilized to reduce anthropogenic carbon oxide emissions.

The following description provides specific details, such as catalyst types, stream compositions, and processing conditions (e.g., temperatures, pressures, flow rates, etc.) in order to provide a thorough description of embodiments of the present disclosure. However, a person of ordinary skill in the art will understand that the embodiments of the present disclosure may be practiced without employing these specific details. Indeed, the embodiments of the present disclosure may be practiced in conjunction with conventional systems and methods employed in the industry. In addition, only those process components and acts necessary to understand the embodiments of the present disclosure are described in detail below. A person of ordinary skill in the art will understand that some process components (e.g., pipelines, line filters, valves, temperature detectors, flow detectors, pressure detectors, and the like) are inherently disclosed herein and that adding various conventional process components and acts would be in accord with the present disclosure. The drawing accompanying the present application is for illustrative purposes only, and is not meant to be actual views of any particular material, device, or system. Additionally, elements common between figures may retain the same numerical designation.

As used herein, the term “lower hydrocarbon” means and includes an aliphatic hydrocarbon having from one carbon atom to eight carbon atoms (e.g., methane, ethane, ethylene, acetylene, propane, propylene, n-butane, isobutene, butane, isobutene, etc.).

As used herein, the temis “higher hydrocarbon” and “hydrocarbon product” mean and include an aliphatic or cyclic hydrocarbon having at least one more carbon atom than a lower hydrocarbon used to form the higher hydrocarbon.

As used herein, the term “aliphatic hydrocarbon” means and includes a saturated or unsaturated, linear or branched hydrocarbon, such as an alkane, an alkene, or an alkyne. The aliphatic hydrocarbon may include only carbon and hydrogen, or may include carbon, hydrogen, and at least one heteroatom.

As used herein, the term “cyclic hydrocarbon” means and includes at least one closed ring hydrocarbon, such as an alicyclic hydrocarbon, an aromatic hydrocarbon, or a combination thereof. The cyclic hydrocarbon may include only carbon and hydrogen, or may include carbon, hydrogen, and at least one heteroatom.

As used herein, the term “heteroatom” means and includes an element other than carbon and hydrogen, such as oxygen (O), nitrogen (N), or sulfur (S).

As used herein, relational terms, such as “first,” “second,” “top,” “bottom,” “upper,” “lower,” “over,” “under,” etc. are used for clarity and convenience in understanding the disclosure and accompanying drawing and does not connote or depend on any specific preference, orientation, or order, except where the context clearly indicates otherwise.

As used herein, the term “substantially” in reference to a given parameter, property, or condition means and includes to a degree that one of ordinary skill in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “and/or” includes any and all combinations of one or more of the associated listed items.

As used herein, the singular forms “a,” “an,” and “the” are intended to include the plural forms as well, unless the context clearly indicates otherwise.

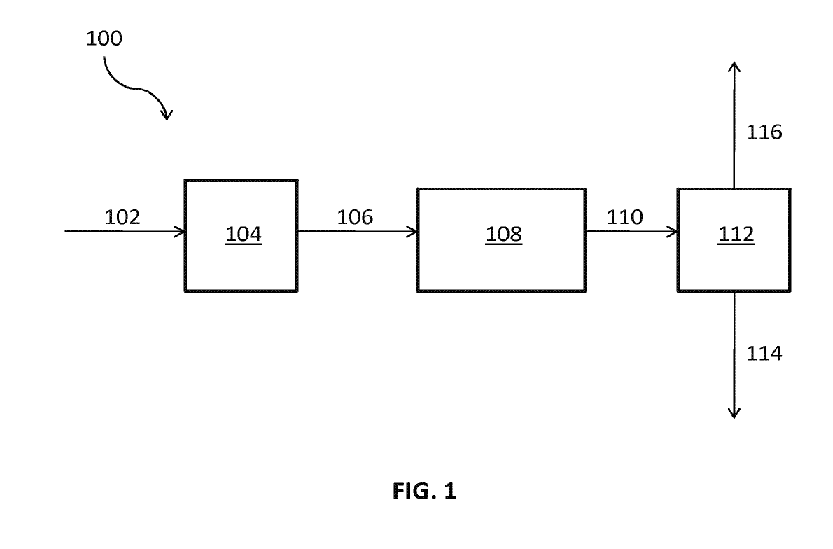

One embodiment of the disclosure will now be described with reference to FIG. 1, which schematically illustrates a hydrocarbon production system 100. As shown in FIG. 1, the hydrocarbon production system 100 may include at least one heating system 104, at least one reactor 108, and at least one separator 112. The heating system 104 may receive at least one source gas stream 102 and may increase the temperature thereof to form at least one heated source gas stream 106. The heated source gas stream 106 exits the heating system 104 and may be delivered into the reactor 108. The reactor 108 converts components of the heated source gas stream 106 to form at least one reaction product stream 110. The reaction product stream 110 exits the reaction 108 and may be directed into the separator 112. The separator 112 separates components of the reaction product stream 110 to form at least one higher hydrocarbon product stream 114 and at least one tail gas stream 116.

The source gas stream 102 may include at least one carbon oxide (e.g., one or more of carbon dioxide and carbon monoxide) and at least one lower hydrocarbon. The carbon oxide may be obtained from the combustion of a primary hydrocarbon, may be obtained from well gases, may be obtained from the atmosphere (e.g., air), or may be obtained from some other source. In some embodiments, the carbon oxide is a combination of carbon monoxide (CO) and carbon dioxide (CO₂). In additional embodiments, the carbon oxide is substantially CO₂. In further embodiments, the carbon oxide is substantially CO. The lower hydrocarbon may be a C₁ to C₈ hydrocarbon that may undergo a chemical reaction with the carbon oxide in the presence of at least one catalyst within the reactor 106 to form the higher hydrocarbon, as described in further detail below. In some embodiments, the lower hydrocarbon is a C₁ to C₈ alkane, such as methane, ethane, propane, or a butane. The source gas stream 102 may, optionally, include other materials, such as hydrogen (H₂), and various other gases (e.g., well gases, nitrogen, etc.). In additional embodiments, the carbon oxide and the lower hydrocarbon may be provided to the heating system 104 in separate streams (e.g., at least one carbon oxide stream, and at least one lower hydrocarbon stream). A ratio of the carbon oxide to the lower hydrocarbon may be selected based on a desired higher hydrocarbon product, as described in further detail below.

The heating system 104 includes at least one apparatus or device configured and operated to increase the temperature of the source gas stream 102 (or separate carbon oxide and lower hydrocarbon streams) to at least one predetermined set point. The apparatus may, for example, comprise at least one of a heat exchanger (e.g., a recuperative heat exchanger, such as a shell-and-tube heat exchanger), and a heater (e.g., a combustion heater, an electrical resistance heater, an inductive heater, an electromagnetic heater, or a combination thereof). The predetermined set point may at least partially depend on operating parameters of the reactor 108, as described in further detail below. For example, the heating system 104 may increase the temperature of the source gas stream 102 up to an operating temperature of the reactor 108, such as a temperature within a range of from about 400° C. to about 1200° C., from about 500° C. to about 1200° C., or from about 650° C. to about 1000° C. The heated source gas stream 106 may exit the heating system 104, and may be directed into the reactor 108. In embodiments where the heating system 104 receives separate carbon oxide and lower hydrocarbon streams, the separate carbon oxide and lower hydrocarbon streams may be combined within the heating system 104 to form the heated source gas stream 106. Alternatively, the separate carbon oxide and lower hydrocarbon streams may remain separate within the heating system 104, and may be directed into the reactor 108 as separate heated carbon oxide and heated lower hydrocarbon streams. The separate heated carbon oxide and heated lower hydrocarbon streams may be heated to the same predetermined set point, or may be heated to separate predetermined set points. In some embodiments, the heating system 104 includes at least one recuperative heat exchanger configured and operated to transfer heat from the reaction product stream to the source gas stream 102.

The reactor 108 may be at least one suitable device or apparatus configured and operated to form the reactant product stream 108 from components or reactants (e.g., the carbon oxide, the lower hydrocarbon, H₂, etc.) of the heated source gas stream 106 (or from components of the separate heated carbon oxide and heated lower hydrocarbon streams). Suitable configurations and operating parameters of the reactor 108 are described in further detail below. The reaction product stream 110 includes at least one higher hydrocarbon. In some embodiments, the at least one higher hydrocarbon includes at least one aliphatic hydrocarbon, such as at least one alkane. In additional embodiments, the at least one higher hydrocarbon includes at least one cyclic hydrocarbon. In yet additional embodiments, the at least one higher hydrocarbon includes a mixture of one or more aliphatic hydrocarbon(s) and one or more cyclic hydrocarbon(s). The reaction product stream 110 may also include at least one additional material, such as one or more of additional reaction products (e.g., water), unreacted components of the heated source gas stream 106 (e.g., CO₂, CO, one or more lower hydrocarbons, H₂, etc.), and a catalyst. The at least one additional material may be separated from the at least one higher hydrocarbon, or may be recovered with the at least one higher hydrocarbon, as described in further detail below.

In some embodiments, the reactor 108 may be configured to produce the at least one higher hydrocarbon from the components of the heated source gas stream 106 by way of at least one of a dehydrocyclization process and a methanation/ethanation process. By way of non-limiting example, the reactor 108 may convert CH₄ to at least one higher hydrocarbon through one or more of the following dehydrocyclization reactions:

2CH_{4(g)}⇄C₂H_{4(g)}+2H_{2(g)} (3)

6CH_{4(g)}⇄C₆H_{6(g)}+9H_{2(g)} (4)

10CH_{4(g)}⇄C₁₀H_{8(g)}+16H_{2(g)} (5).

In addition, CO₂ provided into and/or produced within the reactor 108 may be reacted with H₂ provided into or produced within the reactor 108 to form at least one of CH₄ and ethane (C₂H₆) through one or more of the following methanation/ethanation reactions:

CO_{2(g)}+4H_{2(g)}⇄CH_{4(g)}+2H₂O_{(g)} (6)

2CO_{2(g)}+7H_{2(g)}⇄C₂H_{6(g)}+4H₂O_{(g)} (7).

CO₂ provided into and/or produced within the reactor 108 may also improve catalyst activity by reacting with coke or other carbonaceous deposits that may form on the catalyst through the following reaction:

CO_{2(g)}+coke₍ₛ₎⇄2CO_{(g)} (8).

Water (H₂O) provided into and/or produced within the reactor 108 (e.g., through at least one of Reactions 6 and 7) may also improve catalyst activity by reacting with coke or other carbonaceous deposits that may form on the catalyst through the following reaction:

2H₂O_{(g)}+coke₍ₛ₎⇄2CO_{(g)}+2H_{2(g)} (9).

H₂O provided into and/or produced within the reactor 108 (e.g., through at least one of Reactions 6 and 7) may also react with CH₄ through the following wet methane reformation reaction:

CH_{4(g)}+H₂O_{(g)}⇄CO_{(g)}+3H_{2(g)} (10).

In addition, H₂O provided into and/or produced within the reactor 108 may be at least partially removed from the reaction product stream 110 using the separator 112. In further embodiments, CO provided into and/or produced within the reactor 108 may form at least one additional higher hydrocarbon by reacting with H₂ provided into and/or produced within the reactor 108 through a Fischer-Tropsch process. For example, CO and H₂ may produce CH₄ and H₂O through the following Fischer-Tropsch reaction:

CO_{(g)}+3H_{2(g)}⇄CH_{4(g)}+H₂O_{(g)} (11).

Reactions within the reactor 108 may occur simultaneously (e.g., a single-step reaction process), or may occur consecutively (e.g., a multi-step reaction process, such as a process wherein different reactions are performed within at least one of different reaction vessels and different reaction regions by modifying conditions within the at least one of the different reaction vessels and different reaction regions).

Amounts of the carbon oxide and the lower hydrocarbon within the reactor 108 may be substantially stoichiometric. That is, an amount of the carbon oxide and an amount of the lower hydrocarbon in the reactor 108 may be controlled so that the carbon oxide and the lower hydrocarbon are substantially reacted or consumed within the reactor 108. Alternatively, the amounts of the carbon oxide and the lower hydrocarbon within the reactor 108 may be substantially non-stoichiometric. That is, the amount of the carbon oxide and the amount of the lower hydrocarbon in the reactor 108 may be controlled so that at least a portion of the carbon oxide or the lower hydrocarbon is not reacted or consumed within the reactor 108. A molar ratio of the carbon oxide (e.g., CO₂) to the lower hydrocarbon (e.g., CH₄) in the reactor 108 may, for example, be within a range of from about 1:1 to about 1:10 or higher, such as from about 1:3 to about 1:10, from 1:3 to about 1:5, or about 1:4. The molar ratio of the carbon oxide to the lower hydrocarbon in the reactor 108 may at least partially determine the type of higher hydrocarbon produced. An amount of H₂ in the reactor 108 may also at least partially determine the type of higher hydrocarbon produced. In embodiments where the heated source gas stream 106 is delivered into the reactor 108, the amounts of the carbon oxide, the lower hydrocarbon, and H₂ within the reactor 108 may be controlled by controlling one or more of the amounts of the carbon oxide, the lower hydrocarbon, and H₂ within the heated source gas stream 106. In embodiments where separate heated carbon oxide and heated lower hydrocarbon streams are delivered into the reactor 108, a flow rate of each of the heated carbon oxide stream and the heated lower hydrocarbon stream may be controlled to control the amounts of the carbon oxide, the lower hydrocarbon, and H₂ within the reactor 108.

The reactor 108 includes at least one catalyst. As used herein, the term “catalyst” means and includes any material catalyzing the formation of the higher hydrocarbon from the carbon oxide and the lower hydrocarbon. The catalyst may accelerate reaction rates within the reactor 108, and may also enable the reactor 108 to be operated at lower temperatures. As a non-limiting example, the catalyst may comprise an element of Group 2 (e.g., beryllium, magnesium, calcium, strontium, barium), Group 3 (e.g., scandium, yttrium, lanthanide, actinide), Group 4 (e.g., titanium, zirconium, hafnium), Group 5 (e.g., vanadium, niobium, tantalum), Group 6 (e.g., chromium, molybdenum, tungsten), Group 7 (e.g., manganese, rhenium), Group 8 (e.g., iron, ruthenium, osmium), Group 9 (e.g., cobalt, rhodium, iridium), Group 10 (e.g., nickel, palladium, platinum), Group 11 (e.g., copper, silver, gold), Group 12 (e.g., zinc, cadmium), Group 13 (e.g., boron, aluminium, gallium, indium, thallium), Group 14 (e.g., silicon, germanium, tin, lead), or Group 15 (e.g., arsenic, anotimony, bismuth) of the Periodic Table of Elements, oxides thereof, carbides thereof, alloys thereof, or combinations thereof. The catalyst may, for example, comprise a metal known to be subject to metal dusting. As used herein, the term “metal dusting” refers to a corrosion phenomenon wherein structures formed of and including pure metals and metal alloys degrade (e.g., breakup) into powder or “dust” at temperatures within a range of from about 450° C. to about 850° C. in gaseous environments including carbon. In some embodiments, the catalyst comprises at least one element selected from Groups 5 through 10 of the Periodic Table of Elements.

Various grades of the at least one catalyst may be used. For example, the catalyst may be a grade of an iron-, chromium-, molybdenum-, cobalt-, tungsten-, or nickel-containing alloy or superalloy. Such materials commercially available from numerous sources, such as from Special Metals Corp., of New Hartford, N.Y., under the trade name INCONEL®, or from Haynes, Intl, Inc., of Kokomo, Ind., under the trade name HASTELLOY® (e.g., HASTELLOY® B-2, HASTELLOY® B-3, HASTELLOY® C-4, HASTELLOY® C-2000, HASTELLOY® C-22, HASTELLOY® C-276, HASTELLOY® G-30, HASTELLOY® N, or HASTELLOY® W). In some embodiments, the catalyst is steel. Iron alloys, including steel, may contain various allotropes of iron, including alpha-iron (austenite), gamma iron, and delta-iron. In some embodiments, the catalyst comprises an iron-containing alloy, wherein the iron is not in an alpha phase. As a non-limiting example, the catalyst may comprise at least one of a low chromium stainless steel, steel, and cast iron (e.g., white cast iron). The catalyst may comprise less than or equal to about 22 percent by weight (wt %) chromium, and less than or equal to about 14 wt % nickel (e.g., such as less than or equal to about 8 wt % nickel). In some embodiments, the catalyst comprises 316L stainless steel. 316L stainless steel comprises from about 16 wt % chromium to about 18.5 wt % chromium, and from about 10 wt % nickel to about 14 wt % nickel.

At least a portion of the catalyst may, optionally, be provided within the reactor 108 as a plurality of nanoparticles. As used herein, the term “nanoparticle” means and includes a particle or grain of the catalyst having an average particle diameter of less than about one micron, such as least than or equal to about 500 nanometers (nm). The catalyst nanoparticles may increase the surface area of the catalyst in contact with reactants (e.g., the carbon oxide, and the lower hydrocarbon) within the reactor 108. The catalyst nanoparticles may be monodisperse, wherein all of the catalyst nanoparticles are of substantially the same size, or may be polydisperse, wherein the catalyst nanoparticles have a range of sizes and are averaged. In addition, the catalyst nanoparticles may each have substantially the same shape, or at least some of the catalyst nanoparticles may have a substantially different shape. The catalyst nanoparticles may be stationary within the reactor 108, or may be mobile within the reactor 108. In some embodiments, a portion of the catalyst nanoparticles are stationary within the reactor 108 and another portion of the catalyst nanoparticles are mobile within the reactor 108.

At least some of the catalyst nanoparticles within the reactor 108 may be bound or coupled to solid structures. The reactor 108 may, for example, contain a plurality of catalyst-containing structures each including a catalyst nanoparticle bound to a solid structure, such as a nanofiber. As used herein, the term “nanofiber” means and includes an elongated structure having a cross-section or diameter of less than one micron, such less than or equal to about 500 nm. Nanofibers include structures that are hollow (e.g., nanotubes), and structures that are substantially free of void spaces. The nanofiber may be formed of and include a different material than the catalyst nanoparticle. By way of non-limiting example, one or more of the catalyst-containing structures may include a catalyst nanoparticle bound to a carbon nanofiber, such as a carbon nanotube (e.g., a single-wall carbon nanotube, a multi-walled carbon nanotube, etc.). As another non-limiting example, one or more of the catalyst-containing structures may include a catalyst nanoparticle bound to a nanofiber of another material type, such as a nanotube of boron nitride, boron carbide, aluminum, cadmium sulfide, carbon nitride, titania, silicon, or silicon dioxide. In some embodiments, the reactor 108 includes a plurality of carbon nanotubes each including at least one catalyst nanoparticle bound thereto. The catalyst nanoparticles may, for example, be bound to or embedded within tips (e.g., growth tips) of the carbon nanotubes. The catalyst nanoparticles may be substantially limited to the tips of the carbon nanotubes, or some of the catalyst nanoparticles may be bound to the carbon nanotubes at other locations, such as on sidewalls of the carbon nanotubes. The solid structures may each be of the same material (e.g., carbon) and the same structural type (e.g., nanotubes), or at least one of the solid structures may be of a different material and/or different structural type than at least one other of the solid structures. The catalyst-containing structures may be stationary or may be mobile within the reactor 108.

The solid structures bound to the catalyst nanoparticles may be of any suitable size and shape. For example, if the solid structures include nanofibers (e.g., nanotubes), a length to diameter ratio of each of the nanofibers may be within a range of from about 10,000:1 to about 10:1, such as from about 1000:1 to about 100:1. Each of the solid structures may be of substantially the same size, or at least one the solid structures may be of a different size than than at least one other of the solid structures. In addition, each of the solid structures may be larger than the catalyst nanoparticle bound thereto. The catalyst nanoparticle bound to each solid structure may constitute greater than or equal to about one (1) percent by weight of the catalyst-containing structure (i.e., the combined weight of the solid structure and the catalyst nanoparticle), such as greater than or equal to about five (5) percent by weight, greater than or equal to about ten (10) percent by weight, greater than or equal to about twenty (20) percent by weight, or greater than or equal to about thirty (30) percent by weight. By way of non-limiting example, if the solid structure is a carbon nanotube and the catalyst nanoparticle is platinum, palladium, nickel, or iron, the catalyst nanoparticle may constitute greater than or equal to about ten (10) percent by weight of the catalyst-containing structure.

The catalyst nanoparticles may become bound to the solid structures during the formation of the solid structures. For example, during the formation of nanofibers (e.g., carbon nanotubes), nanoparticles of catalyst may be formed and separated from a bulk catalyst surface and may become bound to or embedded in the nanofibers which grow therefrom (e.g., the nanoparticles of catalyst may be embedded in growth tips of the nanofibers). Suitable methods and systems for forming the solid structures, and hence the catalyst-containing structures, are described in U.S. patent application Ser. No. 13/263,311 and in U.S. Provisional Patent Application No. 61/790,403. As a non-limiting example, catalyst-containing structures including solid carbon of a desired morphology (e.g., carbon nanofibers, such as carbon nanotubes) bound to nanoparticles of catalyst may be formed within at least one of the reactor 108 and another reactor (not shown) using at least one of a Bosch reaction, a Boudouard reaction (i.e., a reduction-oxidation reaction), and a CH₄ reduction reaction. For instance, the catalyst-containing structures may be formed by converting CO₂ and H₂ into solid carbon and H₂O in the presence of a bulk catalyst, according to the following Bosch reaction:

CO_{2(g)}+2H_{2(g)}⇄C₍ₛ₎+H₂O_{(g)} (12),

which may be broken up into two steps, according to the following reactions:

CO_{2(g)}+H_{2(g)}⇄CO_{(g)}H₂O_{(g)} (13)

CO_{(g)}+H_{2(g)}⇄C₍ₛ₎H₂O_{(g)} (14).

In addition, the formation of the catalyst-containing structures through the Bosch reaction may be augmented using disproportionation of CO into solid carbon and CO₂, according to the following Boudouard reaction:

2CO_{(g)}⇄C₍ₛ₎+CO_{2(g)} (15).

Furthermore, the catalyst-containing structures may be formed by converting CO₂ and CH₄ into solid carbon and H₂O in the presence of a bulk catalyst, according to the following CH₄ reduction reaction:

CO_{2(g)}+CH_{4(g)}⇄2C₍ₛ₎+2H₂O_{(g)} (16),

which may be broken up into two steps, according to the following reactions:

CH_{4(g)}CO_{2(g)}⇄2CO_{(g)}²H_{2(g)} (17)

CO_{(g)}+H_{2(g)}⇄C₍ₛ₎H₂O_{(g)} (18).

One or more of Reactions 12 through 18 above may occur simultaneously during the formation of the catalyst-containing structures.

The catalyst-containing structures (e.g., nanofibers including catalyst nanoparticles bound thereto) and the higher hydrocarbon may be formed simultaneously, non-simultaneously, or a combination thereof. In some embodiments, the catalyst-containing structures and the higher hydrocarbon are formed simultaneously within the reactor 108. The components (e.g., carbon oxide, lower hydrocarbon, etc.) of the heated source gas stream 106 (or of the separate heated carbon oxide and heated lower hydrocarbon streams) may be used to form both the catalyst-containing structures and the higher hydrocarbon. In addition, the processing conditions (e.g., temperatures, pressures, flow rates, etc.) used to form the catalyst-containing structures may the same as the processing conditions used to form the higher hydrocarbon. By way of non-limiting example, the catalyst-containing structures and the higher hydrocarbon may be simultaneously formed within the reactor 108 at temperature within a range of from about 400° C. to about 1200° C., such as from about 500° C. to about 1200° C., or from about 650° C. to about 750° C., and at a pressure within a range of from about 9.65×10⁵ pascal (i.e., about 14 pounds per square inch (psi)) to about 6.90×10⁹ pascal (i.e., about 1000000 psi), such as from about 9.65×10⁵ pascal to about 6.90×10⁶ pascal (i.e., about 1000 psi), or from about 1.38×10⁶ pascal (i.e., about 200 psi) to about 4.14×10⁶ pascal (i.e., about 600 psi).

In additional embodiments, some of the catalyst-containing structures may be formed outside the reactor 108 (e.g., in at least one other reactor) simultaneously with the formation of an amount of the higher hydrocarbon within the reactor 108. The catalyst-containing structures formed outside the reactor 108 may then be delivered into the reactor 108 to form an additional amount of the higher hydrocarbon. A portion of the heated source gas stream 106 (or of the separate heated carbon oxide and heated lower hydrocarbon streams) may be used to form the catalyst-containing structures outside the reactor 108, and another portion of the heated source gas stream 106 may be used to form the higher hydrocarbons within the reactor 108. In further embodiments, the heated source gas stream 106 may be used to form the higher hydrocarbons within the reactor 108, and at least one separate stream (not shown) including at least one carbon oxide (e.g., CO, CO₂) and at least one gaseous reducing material (e.g., a lower hydrocarbon, H₂, etc.) may be used to form at least some the catalyst-containing structures outside the reactor 108. The processing conditions used to form the catalyst-containing structures outside the reactor 108 may be substantially the same as or may be different than the processing conditions used to form the higher hydrocarbon with the reactor 108. For example, the catalyst-containing structures formed outside the reactor 108 may be formed at a temperature within a range of from about 400° C. to about 1200° C. (e.g., from about 500° C. to about 1200° C., or from about 650° C. to about 750° C.), and the higher hydrocarbon formed within the reactor 108 may be formed at substantially the same temperature or at a different temperature within the range of from about 400° C. to about 1200° C. (e.g., from about 500° C. to about 1200° C., or from about 650° C. to about 750° C.). In addition, the catalyst-containing structures formed outside the reactor 108 may be formed at a pressure within a range of from about 9.65×10⁵ pascal (i.e., about 14 psi) to about 6.90×10⁹ pascal (i.e., about 1000000 psi), such as from about 9.65×10⁵ pascal to about 6.90×10⁶ pascal (i.e., about 1000 psi), or from about 1.38×10⁶ pascal (i.e., about 200 psi) to about 4.14×10⁶ pascal (i.e., about 600 psi).

In yet additional embodiments, the higher hydrocarbons and at least some of the catalyst-containing structures may be formed non-simultaneously. For example, the catalyst-containing structures may be formed within the reactor 108 prior to the formation of the higher hydrocarbon, and/or may be formed outside the reactor 108 and delivered into the reactor 108 prior to the formation of the higher hydrocarbon therein. The heated source gas stream 106 may be used to form the higher hydrocarbons within the reactor 108, and at least one separate stream (not shown) including at least one carbon oxide (e.g., CO, CO₂) and at least one gaseous reducing material (e.g., a lower hydrocarbon, H₂, etc.) may be used to form at least some the catalyst-containing structures (e.g., the catalyst-containing structures formed outside the reactor 108, the catalyst-containing structures formed inside the reactor 108 before the formation of the higher hydrocarbon, etc.). The processing conditions used to form the catalyst-containing structures prior to the formation of the higher hydrocarbon may be substantially the same as or may be different than the processing conditions used to form the higher hydrocarbon. The catalyst-containing structures may, for example, be formed at a temperature within a range of from about 400° C. to about 1200° C. (e.g., from about 550° C. to about 1200° C., or from about 650° C. to about 750° C.), and the higher hydrocarbon may be formed at substantially the same temperature or at a different temperature within the range of from about 400° C. to about 1200° C. (e.g., from about 500° C. to about 1200° C., or from about 650° C. to about 750° C.). Furthermore, the catalyst-containing structures may be formed at a pressure within a range of from about 9.65×10⁵ pascal (i.e., about 14 psi) to about 6.90×10⁹ pascal (i.e., about 1000000 psi), such as from about 9.65×10⁵ pascal to about 6.90×10⁶ pascal (i.e., about 1000 psi), or from about 1.38×10⁶ pascal (i.e., about 200 psi) to about 4.14×10⁶ pascal (i.e., about 600 psi).

The partial pressure of water (e.g., within the reactor 108, and/or within another reactor) may be utilized to control the formation of the catalyst-containing structures. For example, the partial pressure of water within the reactor 108 may be controlled to form solid structures (e.g., solid carbon) of a desired morphology (e.g., carbon nanofibers, such as carbon nanotubes), and to control the kinetics of solid structure (e.g., solid carbon) formation. Changing the partial pressure of water within the reactor 108 may change carbon activity (A_{c}) within the reactor 108 (and/or within another reactor utilized to form the catalyst containing structures). Without being bound to any particular theory, carbon activity (A_{c}) is believed to be a metric for determining which allotrope of solid carbon will be formed under particular reaction conditions (e.g., temperature, pressure, reactants, concentrations). For example, higher carbon activity may result in the formation of carbon nanotubes, and lower carbon activity may result in the formation of graphitic forms of solid carbon. Carbon activity for a reaction forming solid carbon from gaseous reactants can be defined as the reaction equilibrium constant times the partial pressure of gaseous products, divided by the partial pressure of reactants. For example, in the reaction, CO_{(g)}+H_{2(g)}≈C₍ₛ₎+H₂O_{(g)}, with a reaction equilibrium constant of K, the carbon activity A_{c} is defined as K·(P_{CO}·P_{H2}/P_{H2O}). Thus, A_{c} is directly proportional to the partial pressures of CO and H₂, and inversely proportional to the partial pressure of H₂O. Higher P_{H2O} may inhibit CNT formation. The carbon activity of this reaction may also be expressed in terms of mole fractions and total pressure: A_{c}=K·P_{T}(Y_{CO}·Y_{H2}/Y_{H2O}), where P_{T} is the total pressure and Y is the mole fraction of a species. Carbon activity may vary with temperature because reaction equilibrium constants vary generally with temperature. Carbon activity also varies with total pressure for reactions in which a different number of moles of gas are produced than are consumed. Mixtures of solid carbon allotropes and morphologies thereof can be achieved by varying the catalyst material and the carbon activity of the reaction gases within the reactor 108 (and/or within another reactor utilized to form the catalyst containing structures).

At least some catalyst-containing structures may be formed into a variety of structures suitable for use in different configurations of the reactor 108. For example, if utilized, at least some catalyst-containing structures formed prior to the formation of the higher hydrocarbon may be subjected to one or more of extrusion, powder agglomeration, and compaction (i.e., pressing) processing to form at least one larger structure of a desired shape for use in forming the higher hydrocarbon. In some embodiments, the catalyst-containing structures are pelletized and placed within the reactor 108. For example, an agglomerate powder of the catalyst-containing structures may be pressed into pellets, spheres, and/or other shapes suitable for use in different configurations of the reactor 108 (e.g., a packed bed configuration, a fluidized bed configuration, etc.). Heat may also be applied to form the larger structure(s) including the catalyst-containing structures. For example, the catalyst-containing structures may be compressed or extruded, and heated to form a desired pellet shape. Heat may be applied during the compression of the catalyst-containing structures, after the compression of the catalyst-containing structures, or a combination thereof. During the heating process, chemical agents may be added to functionalize the catalyst-containing structures. Forming the larger structure(s) (e.g., pellets) including the catalyst-containing structures may reduce the tendency of the catalyst-containing structures to elutriate in process streams. The larger structure(s) may be sufficiently porous to exhibit a high effective surface area of the catalyst nanoparticles (e.g., an effective surface area that is not substantially decreased as compared to the catalyst-containing structures alone).

One or more of the catalyst-containing structures (e.g., non-pelletized forms of the catalyst-containing structures) and structures including the catalyst-containing structures (e.g., pelletized forms of the catalyst-containing structures) may, optionally, be at least partially coated with additional catalyst. The additional catalyst may, for example, be deposited on the catalyst-containing structures using conventional deposition processes, such as atomic layer deposition (ALD), chemical vapor deposition (CVD), physical vapor deposition (PVD), electroplating, and metal (i.e., catalyst) carbonyl decomposition, which are not described in detail herein. The additional catalyst may be the same as or different than the catalyst previously described. Coating the catalyst-containing structures (or the structures including the catalyst-containing structures) with the additional catalyst may increase the catalytically effective surface area of the catalyst-containing structures, which may increase conversion efficiency (i.e., the efficiency of converting the lower hydrocarbon and the carbon oxide to the higher hydrocarbon) and facilitate enhanced production of one or more higher hydrocarbon(s).

The reactor 108 may be a continuous reactor or a batch reactor including the catalyst. The reactor 108 may be configured and operated to increase the exposed surface area of the catalyst (e.g., the nanoparticles of catalyst) to at least the carbon oxide and the lower hydrocarbon. The reactor 108 may, for example, be configured and operated to present the catalyst (e.g., pelletized and/or non-pelletized forms of the catalyst-containing structures) as a fixed bed, a moving bed, or a fluidized bed. In some embodiments, the reactor 108 may be a tube or pipe (e.g., a stainless steel tube or pipe) at least partially packed with a non-pelletized form or a pelletized form of the catalyst-containing structures previously described. In additional embodiments, the reactor 108 may be a tube or pipe including the catalyst-containing structures (e.g., carbon nanotubes including nanoparticles of the catalyst bound thereto) attached to a bulk form of the catalyst (e.g., a wafer, cylinder, plate, sheet, sphere, pellet, or other shape of the catalyst). The reactor 108 may be configured and operated to substantially retain the catalyst while enabling the higher hydrocarbon, additional reaction products, and unreacted reactants to exit the reactor 108 in the reaction product stream 110. Alternatively, the reactor 108 may be configured and operated such that at least some of the catalyst (e.g., catalyst nanoparticles bound to solid structures, such as nanofibers, within the reactor 108) exits the reaction 108 with the higher hydrocarbon, additional reaction products, and unreacted reactants. Catalyst exiting the reactor 108 may be conventionally separated from the higher hydrocarbon, additional reaction products, and unreacted reactants, and recycled to the reactor 108 for use in producing additional higher hydrocarbon(s).

Although depicted as a single unit in FIG. 1, one of skill in the art will appreciate that the reactor 108 may include any number of reaction vessels and reaction regions (e.g., reaction zones, reaction chambers, etc.). That is, the reactor 108 may include a single reaction vessel with a single reaction region, or the reactor 108 may include at least one of a plurality of reaction vessels and a plurality of reactor regions. One reaction vessel may, for example, operate under conditions (e.g., temperatures, pressures, etc.) favorable to a first step of a reaction, and another reaction vessel may operate under conditions favorable to a second step of a reaction. Each reaction vessel may be configured and operated to facilitate a reaction step. By way of non-limiting example, a first reaction vessel may be configured and operated to produce a first higher hydrocarbon from the components of the heated source gas stream 106, and a second reaction vessel may be configured and operated to produce a second higher hydrocarbon from one or more of the first hydrocarbon, additional reaction products from the first reaction vessel, and unreacted components of the heated source gas stream 106. As an additional non-limiting example, a reaction vessel may be configured and operated to produce a higher hydrocarbon from the components of the heated source gas stream 106 under a one set of conditions, and another reaction vessel may be configured and operated to produce the higher hydrocarbon from components of the heated source gas stream 106 under a different set of conditions. In addition, if a plurality of reaction vessels are utilized, each the reaction vessels may be independently configured and operated to form and/or receive the catalyst-containing structures previously described. By way of non-limiting example, a first reaction vessel may be configured and operated to form and/or receive at least some of the catalyst-containing structures (e.g., non-pelletized forms of the catalyst-containing structures, pelletized forms of the catalyst-containing structures, or combinations thereof) prior to the formation of the higher hydrocarbon therein, and a second reaction vessel may be configured and operated to form at least some of the catalyst-containing structures simultaneously with the formation of the higher hydrocarbon therein.

The operating temperature of the reactor 108 may at least partially depend on the composition and average particle size of the catalyst therein, and on the higher hydrocarbon desired. Catalyst nanoparticles generally exhibit optimum reaction temperatures at lower temperatures than larger particles of the catalyst. In embodiments where the higher hydrocarbon and the catalyst-containing structures are formed simultaneously within the reactor 108, the operating temperature of the reactor 108 may also at least partially depend a desired morphology of the catalyst-containing structures. By way of non-limiting example, the reactor 108 may have an operating temperature within a range of from about 400° C. to about 1200° C., such as from about 500° C. to about 1200° C., or from about 650° C. to about 750° C. In addition, the formation of the higher hydrocarbon within the reactor 108 may proceed at a wide range of operating pressures. Increasing the operating pressure may increase the reaction rate. The reactor 108 may, for example, have an operating pressure within a range of from about 9.65×10⁵ pascal to about 6.90×10⁹ pascal, such as from about 9.65×10⁵ pascal to about 6.90×10⁶ pascal, or from about 1.38×10⁶ pascal to about 4.14×10⁶ pascal. In embodiments where the reactor 108 includes a plurality of reaction vessels (and/or reaction regions), one reaction vessel may have at least one of a different operating temperature and a different operating pressure than another reaction vessel. For example, different reaction vessels may have different operating temperatures, which may facilitate enhanced production of one or more higher hydrocarbon(s) by way of the same reaction mechanism or different reaction mechanisms. The different operating temperatures of the different reaction vessels may also facilitate enhanced production of one or more catalyst-containing structures (e.g., nanofibers bound to catalyst nanoparticles) by way of the same reaction mechanism or different reaction mechanisms.

A residence time within the reactor 108 may be within a range of from about 1×10⁻⁴ second to about 1×10⁴ seconds, such as from about 1×10⁻³ second to about 100 seconds, or from about 0.01 second to about 5 seconds. The residence time in the reactor 108 may be at least partially controlled by one or more forces (e.g., gravitational forces, electromagnetic forces, centrifugal forces, etc.). In embodiments where the reactor 108 includes a plurality of reaction vessels (and/or reaction regions), a residence time in each of the reaction vessels may be substantially the same, or a residence time in at least one of the reaction vessels may be different than a residence time in at least one other of the reaction vessels.

With continued reference to FIG. 1, the reaction product stream 110 including the higher hydrocarbon exits the reactor 110 and may be directed into the separator 112. The reaction product stream 110 may be substantially gaseous, may be substantially liquid, or may be a multi-phase stream including at least two of a gaseous component, a liquid component, and a solid component. In some embodiments, the reaction product stream 110 is substantially gaseous. The separator 112 may be at least one device or apparatus configured and operated to separate or remove the higher hydrocarbon of the reaction product stream 110 from additional components or materials (e.g., unreacted lower hydrocarbon, unreacted carbon oxide, gaseous H₂O, H₂, catalyst-containing structures, etc.) of the reaction product stream 110. The separator 112 may, for example, be a condensing unit configured and operated to cool the reaction product stream 110 and liquefy the higher hydrocarbon(s) therein. In some embodiments, the separator 112 may be configured and operated to liquefy any gaseous components of the reaction product stream 110 with a boiling point higher than methane, while gaseous components with a boiling point less than or equal to that of methane remain in a gaseous state.

A higher hydrocarbon product stream 114 including the higher hydrocarbon of the reaction product stream 110 exits the separator 112 and may be utilized as desired. In embodiments where the higher hydrocarbon product stream 114 includes a plurality of higher hydrocarbons, the higher hydrocarbon product stream 114 may be subjected to one or more additional separation processes (e.g., liquid separation processes, such as fractional distillation, steam distillation, vacuum distillation, flash evaporation, catalytic cracking, etc.) to separate at least some of the different higher hydrocarbons. The additional separation process may be performed by at least one suitable device or apparatus operatively associated with the separator 112. In some embodiments, at least one component of the higher hydrocarbon product stream 114 may be utilized to produce one or more of an additional amount of the higher hydrocarbon and at least one different higher hydrocarbon. By way of non-limiting example, a portion of the higher hydrocarbon product stream 114 may be pyrolyzed to produce additional H₂ and lower hydrocarbons that may be recycled (not shown), in total or in part, into at least one of the source gas stream 102, the heating system 104, the heated source gas stream 106, and the reactor 108 to produce the additional amount of the higher hydrocarbon (e.g., by way of a combination of Reactions 1 through 6 above). Energy to facilitate the pyrolysis may, for example, be obtained from clean or renewable energy sources, such as solar power, geothermal power, hydroelectric power, wind power, or nuclear power.

A tail gas stream 116 including the additional components of the reaction product stream 110 also exits the separator 112, and may be utilized or disposed of as desired. In some embodiments, at least one component of the tail gas stream 116 may be utilized to produce one or more of an additional amount of the higher hydrocarbon, at least one different higher hydrocarbon, and additional catalyst-containing structures. For example, the tail gas stream 116 may be recycled (not shown), in total or in part, into at least one of the source gas stream 102, the heating system 104, the heated source gas stream 106, and the reactor 108. The tail gas stream 116 may be treated (dried, heated, etc.) prior to recycle. Treating the tail gas stream 116 prior to recycle may at least partially remove at least one component of the tail gas stream 116. By way of non-limiting example, the tail gas stream 116 may be treated to remove water therefrom. In some embodiments, the removed water may, in turn, be electrolyzed to produce H₂ that may be recycled (not shown) in total or in part, into at least one of the source gas stream 102, the heating system 104, the heated source gas stream 106, and the reactor 108 to produce the additional amount of the higher hydrocarbon (e.g., by way of a combination of Reactions 1 through 6 above). Energy to facilitate the electrolysis of the removed water may, for example, be obtained from clean or renewable energy sources, such as solar power, geothermal power, hydroelectric power, wind power, or nuclear power. If present, catalyst (e.g., catalyst nanoparticles included in catalyst-containing structures) separated from the higher hydrocarbon may also be utilized (e.g., recycled to the reactor 108) to produce one or more of an additional amount of the higher hydrocarbon, at least one different higher hydrocarbon, and additional catalyst-containing structures.

1. A method of forming a hydrocarbon product comprising reacting at least one carbon oxide and at least one lower hydrocarbon in the presence of a plurality of catalyst-containing structures each comprising a nanofiber bound to at least one catalyst nanoparticle to form at least one higher hydrocarbon.

2. The method of claim 1, wherein reacting at least one carbon oxide and at least one lower hydrocarbon in the presence of a plurality of catalyst-containing structures comprises reacting the least one carbon oxide, the at least one lower hydrocarbon, and hydrogen gas in the presence of the plurality of catalyst-containing structures.

3. The method of claim 1, wherein reacting at least one carbon oxide and at least one lower hydrocarbon in the presence of a plurality of catalyst-containing structures comprises reacting the least one carbon oxide and the at least one lower hydrocarbon in the presence of the plurality of catalyst-containing structures to form the at least one higher hydrocarbon and water.

4. The method of claim 1, wherein reacting at least one carbon oxide and at least one lower hydrocarbon in the presence of a plurality of catalyst-containing structures comprises reacting the least one carbon oxide and the at least one lower hydrocarbon at a temperature within a range of from about 500° C. to about 1200° C.

5. The method of claim 1, wherein reacting at least one carbon oxide and at least one lower hydrocarbon in the presence of a plurality of catalyst-containing structures comprises reacting the at least one carbon oxide and the at least one lower hydrocarbon at a pressure within a range of from about 9.65×10⁵ Pascals to about 6.90×10⁹ Pascals.

6. (canceled)

7. The method of claim 1, wherein reacting at least one carbon oxide and at least one lower hydrocarbon in the presence of a plurality of catalyst-containing structures comprises reacting carbon dioxide and methane in the presence of the plurality of catalyst-containing structures.

8. The method of claim 1, wherein reacting at least one carbon oxide and at least one lower hydrocarbon in the presence of a plurality of catalyst-containing structures comprises supplying the at least one carbon oxide and the at least one lower hydrocarbon at a molar ratio within a range of from about 1:3 to about 1:10.

9. (canceled)

10. The method of claim 1, wherein the at least one catalyst nanoparticle comprises a metal selected from Groups 5 through 10 of the Periodic Table of Elements.

11. The method of claim 1, wherein the nanofiber of at least some of the plurality of catalyst containing structures comprises a nanotube comprising carbon, boron nitride, boron carbide, aluminum, cadmium sulfide, carbon nitride, titania, silicon, or silicon dioxide.

12. The method of claim 1, wherein the nanofiber of each of the plurality of catalyst containing structures comprises a carbon nanotube.

13. The method of claim 1, wherein reacting at least one carbon oxide and at least one lower hydrocarbon in the presence of a plurality of catalyst-containing structures comprises reacting at least one carbon oxide and at least one lower hydrocarbon in the presence of a plurality of structures each formed from a portion of the plurality of catalyst-containing structures.

14. A method of forming a hydrocarbon product, comprising:
heating at least one carbon oxide and at least one lower hydrocarbon to a temperature within a range of from about 500° C. to about 1200° C. to form at least one heated carbon oxide and at least one heated lower hydrocarbon;
reacting the at least one heated carbon oxide and the at least one heated lower hydrocarbon in the presence of a catalyst to form a reaction product stream comprising at least one higher hydrocarbon; and
separating the at least one higher hydrocarbon from other components of the reaction product stream to form a product stream comprising the at least one higher hydrocarbon and a tail gas stream comprising the other components.

15. The method of claim 14, wherein reacting the at least one heated carbon oxide and the at least one heated lower hydrocarbon in the presence of a catalyst comprises reacting the at least one heated carbon oxide and the at least one heated lower hydrocarbon in the presence of a bulk catalyst to simultaneously form the at least one higher hydrocarbon and catalyst-containing structures each comprising a nanofiber bound to at least one catalyst nanoparticle.

16. The method of claim 14, wherein reacting the at least one heated carbon oxide and the at least one heated lower hydrocarbon in the presence of a catalyst comprises reacting the at least one heated carbon oxide and the at least one heated lower hydrocarbon in the presence of catalyst-containing structures each comprising a nanofiber bound to at least one catalyst nanoparticle.

17-20. (canceled)

21. The method of claim 14, further comprising recycling at least a portion of the tail gas stream to form an additional amount of the at least one higher hydrocarbon.

22. The method of claim 21, wherein recycling at least a portion of the tail gas stream comprises:
treating the at least a portion of the tail gas stream to at least partially remove at least one of the other components; and
recycling remaining components of the at least a portion of the tail gas stream to form the additional amount of the at least one hydrocarbon.

23. (canceled)

24. A system for forming a hydrocarbon product, comprising:
at least one heating system configured to increase the temperature of at least one carbon oxide and at least one lower hydrocarbon to a temperature within a range of from about 500° C. to about 1200° C. to form at least one heated carbon oxide and at least one heated lower hydrocarbon;
at least one reactor configured to convert the at least one heated carbon oxide and the at least one heated lower hydrocarbon in the presence of a plurality of catalyst-containing structures each comprising a nanofiber bound to at least one catalyst nanoparticle to form at least one higher hydrocarbon; and
at least one separator positioned and configured to separate the at least one higher hydrocarbon from at least one other material.

25-26. (canceled)

27. The system of claim 26, wherein the at least one reactor comprises at least one of a packed bed of the plurality of structures, a moving bed of the plurality of structures, and a fluidized bed of the plurality of structures.

28. (canceled)

29. The system of claim 24, wherein at least one of the plurality of catalyst-containing structures comprises carbon nanotube bound to at least one nanoparticle comprising at least one of vanadium, niobium, tantalum, chromium, molybdenum, tungsten, manganese, rhenium, iron, ruthenium, osmium, cobalt, rhodium, iridium, nickel, palladium, and platinum.

30-31. (canceled)

32. The system of claim 24, wherein the at least one separator is positioned and configured to separate the at least one higher hydrocarbon from water.
